(19) United States
(12) Patent Application Publication
(10) Pub. No.: US 2014/0004199 A1
(43) Pub. Date: Jan. 2, 2014

(54) DRUG CARRIER FOR TUMOR-TARGETED THERAPY, ITS PREPARATION METHOD AND ITS USE

(71) Applicant: Hong Xiao, Chengdu (CN)

(72) Inventor: Hong Xiao, Chengdu (CN)

(73) Assignee: CHENGDU NUOEN BIOTECHNOLY CO., LTD, Chengdu, Sichuan (CN)

(21) Appl. No.: 13/994,680

(22) PCT Filed: Mar. 21, 2012
(86) PCT No.: PCT/CN12/72671

§ 371 Date: Jun. 14, 2013

(30) Foreign Application Priority Data
Mar. 23, 2011 (CN) 201110070735.8

(51) Int. Cl. A61K 47/18; A61K 45/06; A61K 31/713

(52) U.S. Cl. CPC A61K 47/186; A61K 31/713; A61K 45/06
USPC 424/489; 514/785; 514/44 R

(57) ABSTRACT

The present invention discloses a drug carrier with the capability of systemic administration through intravenous injection, its preparation methods and it is used for tumor gene therapy, it is belong to the field of tumor-targeted therapy. The carrier of the present invention is a novel liposome which is composed of DOTAP or its analogue and lecithin or its derivative in molar ratio of 20:(7-13), it can form stable complex with bioactive materials, and can deliver these bioactive material to the targeted cells cultured in vitro or in vivo. The complex of the present invention has a larger packaging capability, and the particle size is greatly reduced, that is optimized to 200 nm and below, in an environment of high serum concentration, it maintains high transfection efficiency. The carrier of the present invention packages DNA of tumor suppressor genes or cell suicide gene by forming complexes which can be specifically delivered into tumor cells in vitro, ex vivo or in vivo for gene therapeutic purposes.

### TECHNICAL FIELD

The present invention belongs to cancer gene therapy technology, particularly involving a carrier for tumor-targeted therapy which is with the capability of systemic administration, its preparation method and its use.

### BACKGROUND OF THE INVENTION

At present, in the field of cancer therapy, because the vast majority of bioactive materials which means cancer treatment drugs lack the capability of delivering themselves into the cells, the direct administration has the shortcoming of low bioavailability, high cytotoxicity, etc. In order to overcome these shortcomings, a large number of carriers have been studied. With better understanding of the molecular mechanism of many diseases at gene level, gene therapy becomes the most promising routes to treat numerous diseases especially malignant tumors, which means that a variety of carriers are used for packaging nucleic acids and other bioactive materials to reach the administration target to transduct and express for targeted cancer therapy. The carrier is generally divided into viral vectors (such as retrovirus and adenovirus) and non-viral vectors (such as cationic polymers and vesicles). Genetically modified virus became the focus in early stage, but experienced safety and efficiency drawback at the start of the century, with that concern, scientific interests in non-viral nanoparticle delivery systems grows rapidly. Current non-viral vector systems can be categorized as cationic liposomes, DNA-polymer conjugates and naked DNA. Of those, gene therapy using cationic liposomes as a carrier vehicle for DNA delivery is of our great interest.

Eukaryotic cells are contained within a bilayer structure formed by bipolar lipids called cell membrane which protects cells by prohibiting passive permeability of chemicals at its surrounding. There are three major classes of cell membrane components: phospholipids, Glycolipids and Cholesterols. All of them can form fatty drops in aqueous solution spontaneously due to their unique bipolar structures. Those fatty structures, named liposome, are closely resemblance of natural cell membrane as visualized first by Dr Bangham and R. W. Home in 1961. Liposomes can fuse with cell membrane and transfer the carried content into cells. Thus, liposomes are broadly used in drug delivery applications, i.e., the aerosol treatment of respiratory diseases. Anti-cancer drugs such as Doxorubicin (Doxil), Camptothecin and Daunorubicin (Daunoxome) are currently being marketed in liposome forms.

With constant studies, People found that DOTAP, named 1,2-bis(olcoyloxy)-3-(trimethylammonio)-propane, and its analogues can transfer DNA into cultured cells by fusing into cell membrane due to the net positive charges added by modification and the similarity of lipid structures. The modification of electric change makes cationic lipid getting closer to negatively charged cell membranes to ensure active fusion between liposome and cell membrane. Unfortunately, this change also invokes immune defense mechanism. Indeed, DOTAP liposome complex uptake by cells is completely knocked down in the presence of serum concentration above 50%.

To solve the problem that DOTAP and its analogues fail to deliver in the presence of serum especially high serum concentration, Cholesterol as helper lipid to overcome serum inhibition was first produced in 1991 by Gao and Huang, who suggested that using cholesterol mixed with DOPE is particularly effective in vivo. Cholesterol stabilizes lipid bilayers which lead to the liposome that has been most frequently used in cystic fibrosis clinical trials. In 1995, Liu etc used liposomes containing cationic lipid dimethyldioctadecyl ammonium bromide (DDAB) in a 1:1 molar ratio with cholesterol to mediate high level chloramphenicol acetyltransferase (CAT) reporter gene expression via systemic injection. Templeton etc, delivered CAT reporter gene into mouse tissues using DOTAP:Cholesterol mixture at molar ratio of 1:1 through intravenous injection in 1997. They found transgene expressed in variety of tissues with majority of transgenes accumulated in mouse lungs and livers. (Templeton etc., 1997)

Serum inhibition effects on cationic liposomal mediated gene transfection is complicated and not completely understood for now. For any delivery system to work in human bloodstream, the physiological serum concentration of 80%-100% is the first barrier to overcome. Cholesterol has proven to be efficient helper lipid to make in vivo delivery possible. Transfecting cultured cells in 80%-100% serum concentration (physiological condition in human bloodstream) can be deployed to replace animal tests in assessment of liposome serum stability and transfection efficiency. Optimization of cationic lipid DNA complexes for transfection efficiency in the presence of high concentrations of serum is used to identify Dotap:Cholesterol complexes with high transduction efficiency in mice.

L-a-Lecithin is a major constituent of cell membranes, and it is without immunogenicity. As the active ingredient in the formation of liposomes, it can form liposome, alone or as a helper lipid. Helper lipid serves important roles for special purposes, like cholesterol protects packed materials from serum degradation, helping target selection etc, but usually has adverse effect on liposome packaging capacity. In addition to a positively charged head, another neutral head groups, DOTAP and L-α-lecithin have very similar hydrophobic chain, to ensure that the two substances can be evenly mixed at any molar ratio. In traditional studies, it added DOTAP to the lecithin to increase transfection efficiency, but in general, as Templeton proved, transfection rate is not high in vivo. By adding a small amount (5%) of DOPS to DOTAP:cholesterol (50:45) to form liposomes of DOTAP:cholesterol:DOPS (50:45:5), in vivo gene expression dropped dramatically-replacing cholesterol by DOPC, in vivo gene expression almost completely disappeared. Many studies for testing liposomes formed by mixing lecithin or its derivatives such as DOPS (Dioleoyl Phosphatidylserine), DOPC (Oleoyl Phosphatidylcholine), DOPE (Dioleoyl phosphatidyl-ethanolamine) with DOTAP at molar ratio of 1:1, they are not suitable for gene transfection in vivo. Mixing L-α-lecithin or its derivative with DOTAP or its analogue as carrier were reported as recent as 2009. One of problems is that the molecule ratio 1:1 of Dotap vs Lecithin were inherently used according to classic cationic lipid and neutral helper lipid model. DOTAP:lecithin in the above study more as a negative control in the application, rather than as a gene carrier candidate. Combination of L-α-lecithin and its derivatives with DOTAP and its analogues has better packaging capability than the combination of DOTAP and cholesterol, but the larger lecithin head groups positively affect liposome complex transfection by hampering the exposure of smaller cationic head of DOTAP as suggested by Templeton etc. In vivo gene transfer was eliminated almost entirely. Besides, the size of liposome complex is dilated well beyond 300 nm due to unfavorable amount of lecithin (Templeton, et al, 2009). Lecithin and its derivatives as helper lipids to improve the transfection efficiency of liposome in the related U.S. patent application had been rejected (eg, U.S. Pat. No. 6,413,544 and No. 6,770,291). Analyzing the reason why the combination of L-alpha-lecithin and its derivatives with DOTAP and its analogues was rejected from the principle of the formation of liposomes, it may due to the inappropriate amount of lecithin added and inappropriate proportion. If we want further improve the targeting of the carrier, serum stability of the carrier after packaging with the drug and bioavailability, we need for further exploration for the proportion of the carrier.

In addition, the size of complex formed by carrier loading bioactive materials is critical for systemic delivery (Uchiyama et al, 1995; Ishida, 1999). For example, liposomes in the range of 100 to 150 nm have been shown to preferentially accumulate in tumors due to the vasculature-enhanced permeability and retention (EPR) effect. The endothelial wall of all healthy human blood vessels is encapsulated by endothelial cells that are bound together by tight junctions. These tight junctions stop any large particle in blood from leaking out of blood vessel. Tumor blood vessels do not contain the same level of seals between cells and are diagnostically leaky. This ability is known as the Enhanced Permeability and Retention effect (EPR effect). Liposomes of certain sizes, typically between 90-250 nm, can rapidly enter tumor sites from the blood, but are kept in the bloodstream by the endothelial wall in healthy tissue vasculature. (Zhang et al, 2008). Particle size of complex prepared by current sound waves and/or extrusion is general above 300 nm, so controlling the size of the carrier and the size of the carrier after loading bioactive materials is very important.

When cationic liposome mixed with DNA, positively charged liposome and negatively charged DNA are attracted to each other due to electrostatic forces. Visible encapsulation transition starts immediately and whole encapsulating process is finished in seconds. The encapsulated liposome DNA complexes swell in sizes during the transition. Plasmid DNA is macromolecule compared to most small molecule drugs. The size of complex partly depends on the size and quantity of the packaged nucleic acid bioactive materials such as DNA, so the control of complex size can not depend on the control of liposome alone. Generally speaking, as DNA concentration increases, the size of complex grows. When DNA concentration surpassed liposome packaging capacity, precipitation happens. For DNA to work effectively at desired therapeutical dosage, more DNA needs to be delivered in a single vesicle, especially in the case of cancer gene therapies or disease suppression therapies. The threshold of DNA dosage requirement for therapeutic effectiveness limits the choice of using less DNA to achieve smaller particle size in most cases. Nanoparticles that are larger than 400 nm in size are not ideal for intravenous injection due to potential cause of embolism in mice leading to animal death. Liposome composition with large packaging capacity and small particles is the ideal formula for success.

Excessive DNA in liposome composition causes enlarged particle sizes and precipitation while insufficient DNA in liposome complex also results in increased toxicity. Properly prepared liposome DNA complexes showed less toxicity than free liposome did. Efficiently packing DNA to the liposome's capacity is practically important not only to maximize transfection efficiency but to reduce liposome-DNA toxicity. At present, the packaging amount of carrier is random and difficult to control due to no method for controlling carrier to maximum packaging amount yet, and then leads to poor treatment.

### SUMMARY OF THE INVENTION

One of technical problems to be solved by the present invention is that disclose a drug (bioactive material) carrier, which can load bioactive material for tumor prevention and therapy, transfect gene targeted and is with better serum stability.

The solution to solve this technical problem is that a drug delivery carrier for tumor-targeted therapy, consisting of two components, which is DOTAP or its analogues and lecithin or its derivatives, the said DOTAP or its analogues and the said lecithin or its derivatives are at molar ratio of 20:(7-13), wherein, the said DOTAP analogues includes but not limit to DOTMA (1,2-di-O-octadecenyl-3-trimethylammonium propane chloride), DDAB (Dimethyldioctadecylammonium Bromide) or methyl sulfate of DOTAP, the following is the structure of said DOTAP:

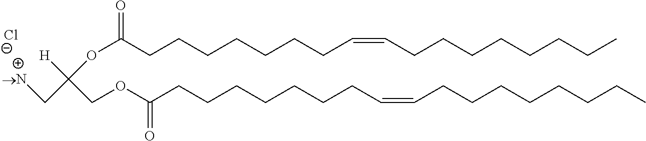

The said lecithin derivatives includes but not limit to DOPC (1,2-dioleoyl-sn-glycero-3-phosphocholine), DOPS (1,2-dioleoyl-sn-glycero-3-phospho-L-serine) or DOPE (1,2-dioleoyl-sn-glycero-3-phosphoethanolamine), the following is the structure of the said lecithin:

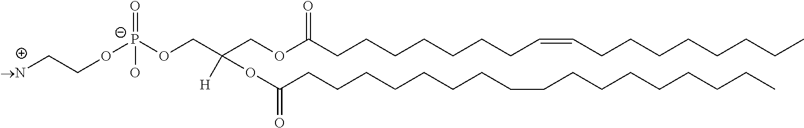

As preferred, the said DOTAP or its analogues and the said lecithin or its derivatives are at molar ratio of 20:(8-11).

Further, the said DOTAP or its analogues and the said lecithin or its derivatives is at molar ratio of 20:9.

The second technical problem to be solved by the present invention is that disclose preparation method of drug carrier for tumor-targeted therapy, it prepares carrier with suitable particle size to meet the needs of drug loading and targeted therapy.

The solution to solve this technical problem is that, prepare according to following steps:

a. mix above-mentioned DOTAP or its analogues and above-mentioned lecithin or its derivatives according to above-mentioned molar ratio with the organic solvent in the container, and then purge the container with nitrogen;

b. heat to 30° C. to remove chloroform by evaporation, and form liposomes film;

c. at 50° C., dissolve the liposomes film from step b with D5W solution;

d filter one time with filter of pore size 0.45 μm, and then filter four times with filter of pore size 0.1 μm, done

The third technical problem to be solved by the present invention is that disclose the use of drug carrier for tumor-targeted therapy.

The solution to solve this technical problem is that apply said carrier in field of tumor therapy.

As preferred, the said carrier form complex by loading negatively charged bioactive materials.

Further, deliver negatively charged bioactive materials into live cells in vitro, ex vivo or in vivo.

Further, the said negatively charged bioactive materials are DNA, RNA or oligomeric nucleic acid.

Further, the said complex is mixed with protein ligands, antibodies or steroids additionally.

Further, the range of particle size range of the said complex is 90 nm-250 nm.

Further, the said complex is used for combination with chemotherapy, radiation therapy or both.

Further, prepare the said complex in form of injection or aerosol.

Further, the maximal packaging capacity of the carrier to load negatively charged bioactive materials is achieved, the method is that, the correction between absorbance of said complex at the wavelength of 260 nm and the concentration of said negatively charged bioactive materials is in line with Sinusoidal Curve Fit model between the carrier and the concentration of said negatively charged bioactive materials; The first peak of sinusoidal curve indicates the maximal packaging capacity of the carrier.

Further, the lowest cytotoxicity is achieved, the method is that the net increase of optical absorbance of the said complex at 260 nm is correlated with cytotoxicity of the complexes; the DNA concentration at the first peak of OD260 sinusoidal curve of the complexes indicates the concentration for lowest cytotoxicity of complexes in vitro and in vivo.

Further, the highest transfection capability is achieved, the method is that: net increase of optical absorbance of the said complex at 260 nm is positively correlated with transfection capability of the complexes; the DNA concentration at the first peak of OD260 sinusoidal curve of the complexes indicates the concentration of highest transgene expression of complexes in vitro and in vivo.

After a large number of experiments carried out by the inventor of the present invention, we found, compared with existing liposome carrier, the present carrier formed by an appropriate proportion of DOTAP or its analogues and lecithin or its derivatives delivers bioactive materials such as DNA, RNA, or oligomeric nucleic acid in vitro or in vivo environment, it have good serum stability, and better targeted transduction. In addition, the use of appropriate preparation methods, the particle size of the complexes made by the carrier disclosed in present invention and the bioactive materials of the present invention are appropriate due to appropriate preparation method and more conductive to drug loading and targeted therapy.

By theoretical analysis and a large number of experiments, the present invention proved that drug carrier made by DOTAP or its analogues and lecithin or its derivatives with an appropriate proportion as well as the appropriate preparation method can have better tumor treatment targeting and lower cytotoxicity, and it has overcome the technical bias that combination of DOTAP or its analogues with lecithin or its derivatives are not suitable for drug carriers, especially the carrier of the gene-based drug therapy of tumor, and it made an unexpected technical effect: the liposome as a carrier loading DNA and or so has better targeting specific and better blood stability.

The study of the present invention found that, structure of lipids and proposed model for DOTAP:Lecithin:DNA complex formation, as shown in FIG. 1, DOTAP lipid and co-lipid Lecithin share the same double chain hydrophobic tail. Cationic DOTAP contains a modified, positively charged head group and Lecithin with natural phosphatyle head group. Liposome shown in the figure is made of the mixture of DOTAP:Lecithin at molecular ratio of 20:9 and filtered by 0.1 um filter to produce dominantly small unilammellar vesicles (SUV). The large head group of lecithin shields smaller positively charged DOTAP to evade serum degradation and increasing liposome complex stability in serum or bloodstream.

DOTAP are distributed in two salt forms (from Avanti Polar Lipids), methyl sulfate or chloride salts. There is no structural difference between methyl sulfate DOTAP and DOTAP other than counter ions used. So DOTAP in both salt forms are interchangeable. The structures of both forms of DOTAP are compared below:

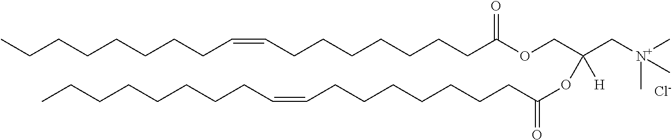 [SMILES: CCCCCCCC/C=C\CCCCCCCC(=O)OCC(C[N+](C)(C)C)OC(=O)CCCCCCC/C=C\CCCCCCCC ; [Cl-]]

DOTAP in Chloride Salt

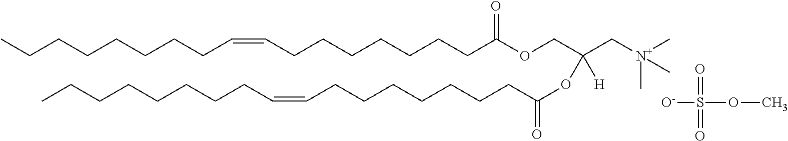

DOTAP in Methyl Sulfate Salt

Lecithin is natural phospholipid and used in current invention to provide dimensionally protective group to shield smaller DOTAP hydrophilic head group from immunologic clearance. DOPC is a synthetic derivative which inherits the same hydrophilic head group as the hydrophilic head of lecithin. The head groups of DOPC can provide the similar protection to DOTAP as lecithin does. The lecithin 16 Carbon saturated fatty chain is replaced with 18 carbons unsaturated fatty chain in DOPC to lower melting point and provide better fluidity in physiological condition. The 18 carbon unsaturated fatty chain of DOPC is the same as DOTAP fatty chains as shown in the picture. DOPC and DOTAP can form seamlessly liposome mixture together. DOPC can be used to replace Lecithin in current invention in preparation of DOTAP:DOPC liposome, the resulting liposome should retain the similar physical and biochemical characteristics as DOTAP:Lecithin liposome. Indeed, DOTAP:DOPC Liposome was prepared successfully by following the same procedures as described for DOTAP:Lecithin liposome. The DOTAP:DOPC liposome displayed the same particle size, packaging capacity and transfection efficiency as its DOTAP:Lecithin liposome counterpart. The Lecithin, DOPC and DOTAP structures are compared as below:

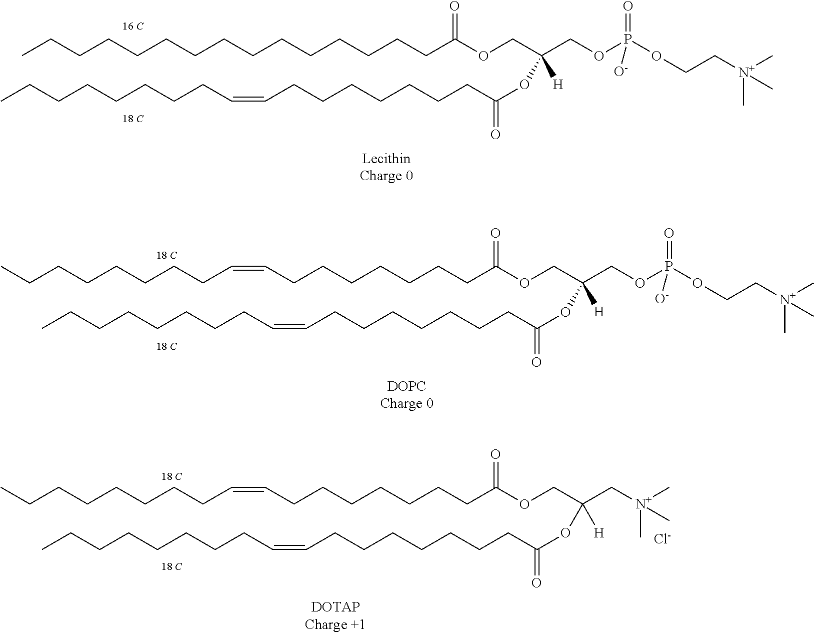

At the same time exploring a better component and proportion of the carrier, the present invention also disclosed the preparation method for the carrier. The preparation method involves dissolving and mixing DOTAP or its analogues with lecithin or its derivatives in organic solvents such as chloroform, evaporation removal of organic solvent to form liposome thin film, hydrolysis of DOTAP or its analogues and lecithin or its derivatives, then produced as homogeneous single unilamillar vesicles (SUV) structure with particle size around 80-100 nm by extrusion filtration with 0.1 um filters for 4 times. The method can be scaled up for large scale manufacture or modified to add ligands for target-specific delivery. The resulting structure is reproducible and stable for at least 2 years at proper storage condition. The said structure is capable of combined with bioactive reagents to form active complex. The carrier of the present invention provides protection against serum degradation and gene transfection ability to bioactive reagents in the form of liposome complexes.

This carrier can be used for tumor therapy. One of these uses is that this carrier can form complex by mixing with DNA, RNA, Oligonucleotide, and so on. Said complex possess the capability of high efficient gene transfer to cultured cells at the presence of high serum concentration or to in vivo human tumors implanted in experimental animals via intravenous administration. The main targets of said complex are fast growing tumor cells in vivo and in vitro. This grants its primary potential in human cancer gene therapy.

The present invention further relates to said complexes which can be optimized between 90 nm to 250 nm with therapeutically relevant dosage. The study indicated that 1 μg/μl DNA, RNA, Oligonucleotide carrying pro-apoptotic genes can be mixed with equal volume of DOTAP:Lecithin liposome to form liposome DNA complexes with particle size around 200 nm.

The present invention also relates to an optimization method for liposome DNA complex formation. Based on a large number of experiments, we found that the measurement of said complex optical absorbance at 260 nm can be used to determine liposome packaging capacity. The OD260 value of said complex (such as DOTAP:Lecithin:DNA liposome complex) is correlated with concentration of bioactive materials such as DNA, RNA, oligonucleotide in sinusoidal model. The first peak of sinusoidal curve indicates the maximal liposome packaging capacity against bioactive materials such as DNA, RNA, oligonucleotide.

The present invention further relates to any kits made by said carrier. The carrier of present invention can be made commercially available alone or as components in a kit for specific application. Our invention also includes the method of OD260 sinusoidal curve model for liposome nucleic acid optimization in any kit, instruction or manual.

The present invention further relates to the use of tumor suppressor genes, in example of tumor suppressor gene p53 mentioned in embodiment of the present invention, to form tumor suppressor gene complex with carrier of the present invention for cancer gene therapy.

The present invention further relates to the use that complexes containing bioactive materials, i.e., nucleic acid can be administered by intravenous, intramuscular, intraperitoneal, subcutaneous intralesional injection or infusion, or by aerosol inhalation.

In summary, due to the above technical solutions, the beneficial effects of the present invention are: the carrier of the present invention by appropriate preparation method and loading nucleic acids or other bioactive materials, which can be used for tumor-targeted therapy by the systemic administration, can be targeted deliver in vitro, ex vivo or in vivo, with ideal effect of maximum packaging volume, targeted transfection gene and serum stability.

### BRIEF DESCRIPTION OF THE DRAWINGS

FIG. 1A is the structure of DOTAP, B is the structure of L-α-Lecithin, C is the formation model of complex of DOTAP:Lecithin:DNA, “−” in C means the negative charge of Nucleic acid, “+” in A means positive charge;

FIG. 2 the schematic that the large head group of natural lecithin shields DOTAP from serum degradation and increasing liposome complex stability in serum and bloodstream;

FIG. 3 Comparison chart of transfection abilities of carrier composed by different proportion of DOTAP vs Lecithin in serum-free medium on H1299 cells;

FIG. 4 DOTAP:Lecithin:DNA complexes transfection efficiency varies with serum concentration;

FIG. 5 the relationship between transfection efficiency of DOTAP:Lecithin:DNA complexes and said particle size, wherein the molar ratio of DOTAP:Lecithin is 20:9;

FIG. 6A-F the relationship chart of OD260 value of DOTAP:Lecithin:DNA complexes vs DNA concentration;

FIG. 7 Transfection Efficiency of complex formed by carrier and pFCB-eGFP differs on cell lines, said carrier is composed by DOTAP and Lecithin with the molar ratio 20:9;

FIG. 8 In vivo tumor LacZ gene expression in lung and liver with or without tumors, wherein, A: Liver from control mouse. B: Liver with metastatic A549 tumors. C: Lung and spleen from control mouse. D: Lung with metastatic A549 tumors;

FIG. 9 Intravenous Administration of DOTAP:Lecithin:pFCB-p53 suppressed tumor growth in subcutaneously implanted A549 tumor model, said DOTAP and Lecithin with the molar ratio 20:9.

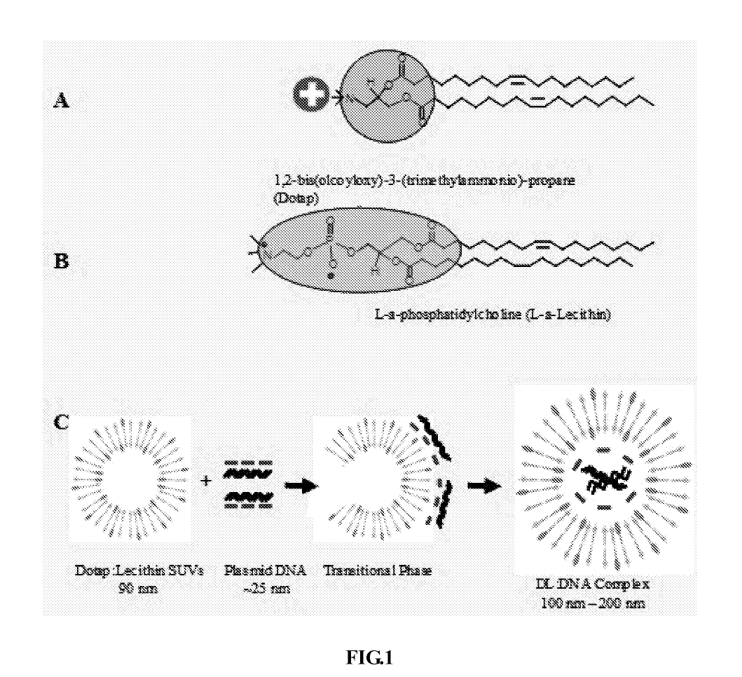

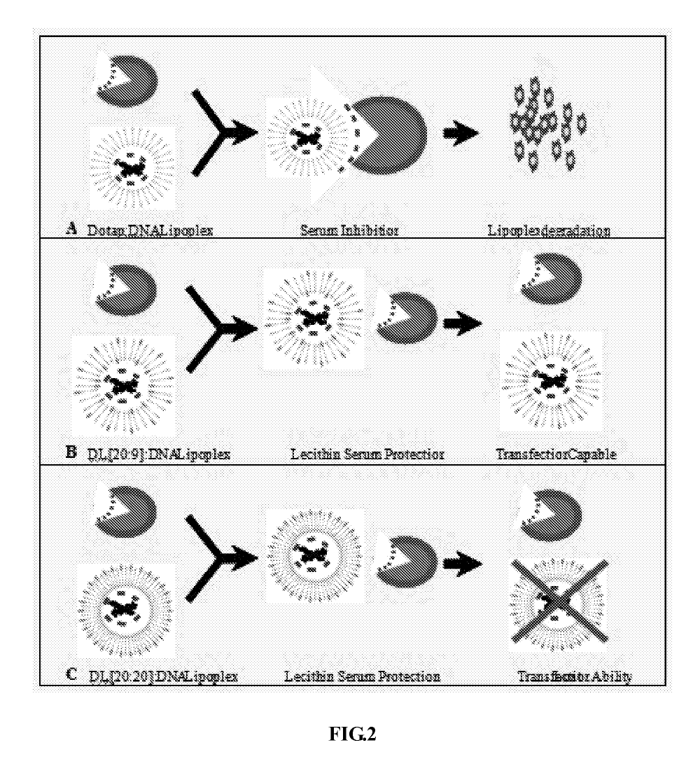

Table 1 Abbreviation used to represent DOTAP, Lecithin concentration ratio;

Table 2: Transfection rate and cytotoxicity comparison, wherein, XTT reading represents the percentage of alive cells in treated groups vs untreated control cells, “Luc” represents that luciferase activities were measured for gene transfer efficiency.

### EMBODIMENT

The following detailed explain the present invention by combining with the drawings,

Although the present invention has been fully described in connection with embodiments thereof with reference to the accompanying drawings, it is to be noted that various changes and modifications will become apparent to those skilled in the art. Such changes and modifications are to be understood as being included within the scope of the present invention as defined by the appended claims.

In embodiment of the present invention:

Table 1 represents the abbreviation used to represent DOTAP, Lecithin concentration ratio

TABLE 1

| | DL[20:8] | DL[20:9] | DL[20:10] | DL[20:11] |
|---|---|---|---|---|
| DOTAP Concentration | 4 mM | 4 mM | 4 mM | 4 mM |
| Lecithin Concentration | 1.6 mM | 1.8 mM | 2 mM | 2.25 mM |
| DOTAP:Lecithin | 20:8 | 20:9 | 20:10 | 20:11 |
| [Molar Ratio] | | | | |

Table 2 is transfection rate and cytotoxicity comparison

| | 10% FBS + | | 100% FBS + | | 100% FBS + | |
|---|---|---|---|---|---|---|
| | 0.25 ul | | 0.5 ul | | 0.25 ul | |
| | Luc | XTT | Luc | XTT | Luc | XTT |
| H1299 | | | | | | |
| 2 mM DL[20:9] | 30.627 | 97 | 14.194 | 96 | 4.998 | 101 |
| 4 mM DL[20:9] | 39.722 | 97 | 23.997 | 97 | 8.853 | 101 |
| 5 mM DL[20:9] | 52.792 | 101 | 30.14 | 96 | 10.631 | 99 |
| 6 mM DL[20:9] | 47.024 | 102 | 33.113 | 95 | 13.789 | 101 |
| 10 mM DL[20:9] | 49.409 | 104 | 48.152 | 97 | 22.723 | 103 |
| 10 mM DC[20:20] | 7.103 | 105 | 6.662 | 105 | 3.389 | 106 |
| A549 | | | | | | |
| 2 mM DL[20:9] | 0.211 | 71 | 0.138 | 97 | 0.048 | 102 |
| 4 mM DL[20:9] | 0.213 | 73 | 0.25 | 99 | 0.084 | 103 |
| 5 mM DL[20:9] | 0.258 | 74 | 0.209 | 95 | 0.084 | 100 |
| 6 mM DL[20:9] | 0.305 | 72 | 0.199 | 100 | 0.077 | 100 |
| 10 mM DL[20:9] | 0.259 | 73 | 0.145 | 102 | 0.071 | 103 |
| 10 mM DC[20:20] | 0.043 | 66 | 0.026 | 95 | 0.012 | 96 |
| NHBE | | | | | | |
| 2 mM DL[20:9] | 0.017 | 48 | 0.004 | 88 | 0.001 | 112 |
| 4 mM DL[20:9] | 0.024 | 57 | 0.009 | 92 | 0.002 | 102 |
| 5 mM DL[20:9] | 0.034 | 64 | 0.007 | 66 | 0.001 | 96 |
| 6 mM DL[20:9] | 0.007 | 55 | 0.007 | 75 | 0.001 | 89 |
| 10 mM DL[20:9] | 0.007 | 39 | 0.005 | 61 | 0.001 | 77 |
| 10 mM DC[20:20] | 0.001 | 21 | 0.002 | 67 | 0.001 | 73 |

### Example 1

### Experiment of Transfection Abilities of Different Proportion of DOTAP vs Lecithin in Serum-Free Medium on H1299 Cells

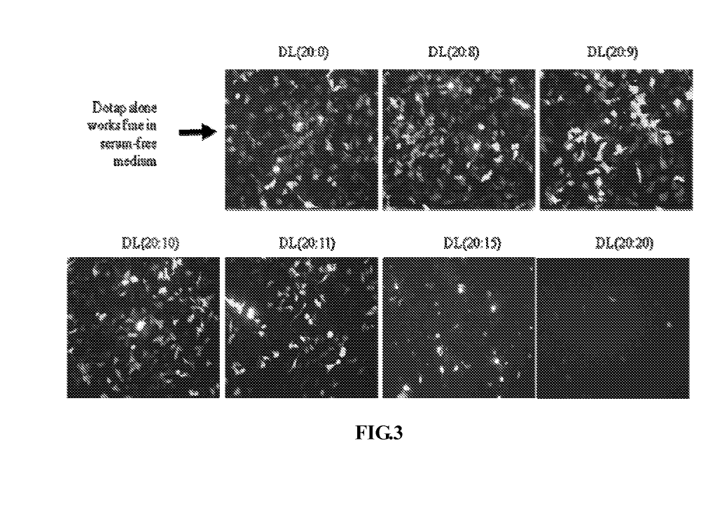

Shown in FIG. 3, Transfection abilities of DOTAP decreased with mounting Lecithin presence in H1299 cells. 1 μg/μl of pFCB-eGFP were mixed with equal volume of DOTAP:Lecithin liposomes in different Dotap:Lecithin ratio. H1299 cells were seeded in 12 well plates and treated by 1 ml of Opti-MEM with 2.5 μl of DOTAP:Lecithin:DNA complex. 4 hours after transfection, medium were replaced by RPMI with 10% FBS and plates returned to incubator for another 48 hours. Pictures were taken under fluorescence microscope at magnificence of 200; said picture is shown in FIG. 3.

### Example 2

### DOTAP:Lecithin:DNA Complexes Transfection Efficiency Varies with Serum Concentration

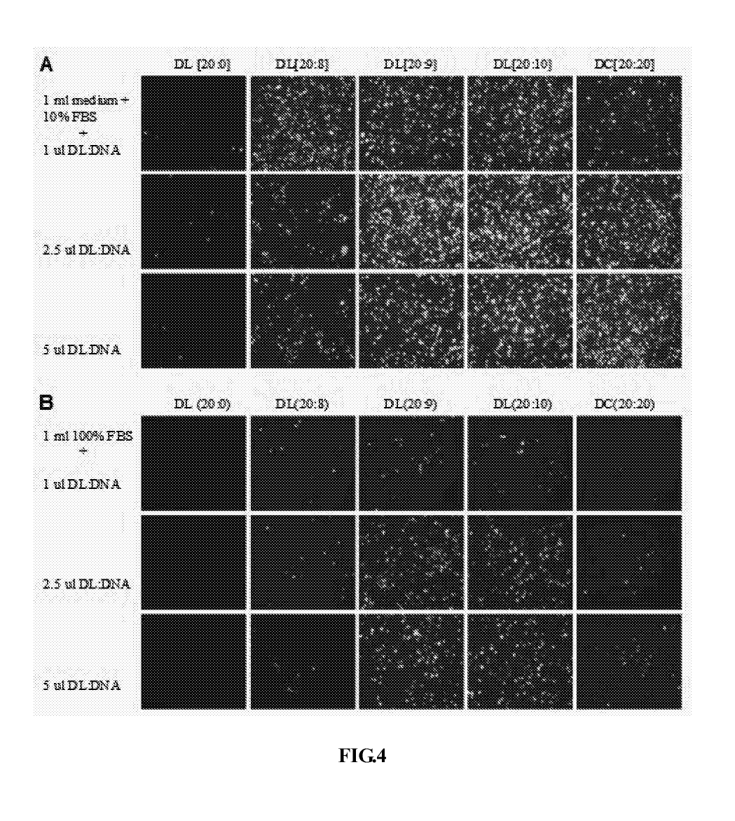

As shown in FIG. 4, H1299 were seeded in 12 well plates and incubated in 37° C. with 5% CO2 overnight. When cells reached 30% confluent, cells were treated with 1 μl, 2.5 μl or 5 μl DOTAP:Lecithin:pFCB-eGFP in 1 ml RPMI with 10% FBS or 1 ml 100% FBS. Treated cells were shaked gently by hand every 30 minutes. The transfection medium was replaced with 1 ml RPMI with 10% FBS after 4 hours. Pictures were taken under fluorescence microscope 48 hours after transfection. 8 mM of DOTAP:Lecithin with different DOTAP:Lecithin molecular ratio were combined with equal volume of 1 μg/μl pFCB-eGFP plasmid DNA. The pictures from DOTAP:Lecithin liposomes with molar ratio ranging 20:8 to 20:10 were shown here. DC was 4 mM DOTAP:cholesterol liposome in place of DOTAP:Lecithin for gene transfection control. Pictures were taken under fluorescence microscope at magnificence of 200; said picture is shown in FIG. 4.

### Example 3

### The Experiment of the Relationship Between Transfection Efficiency of DOTAP:Lecithin:DNA Complexes and Said Particle Size, Wherein the Molar Ratio of DOTAP:Lecithin is 20:9

As shown in FIG. 5, DNA mixture containing 0.2 μg/μl of pFCB-eGFP and 1 μg/μl of pFCB-Luc were mixed with equal volume of DOTAP:Lecithin. Complexes then pass through filters with pore size of 1 um, 0.45 um. The pass-through were marked as 1 um and 0.45 um. DNA mixture containing 0.2 μg/μl of pFCB-eGFP and 0.6 μg/μl of pFCB-Luc were mixed with equal volume of DOTAP:Lecithin. Complexes then passed through filters with pore size of 0.2 um, 0.1 um. The pass-through were marked as 0.2 μm and 0.1 μm. H1299 were seeded in 12 well plates 24 hours before and reached 30% confluence at time of transfection. The transfection was performed in 1 ml of RPMI with 10% FBS or in 100% FBS with either 2 μl or 5 μl DOTAP:Lecithin:DNA complexes.

### Example 4

### The Experiment of the Relationship Chart of OD260 Value of DOTAP:Lecithin:DNA Complexes Vs DNA Concentration

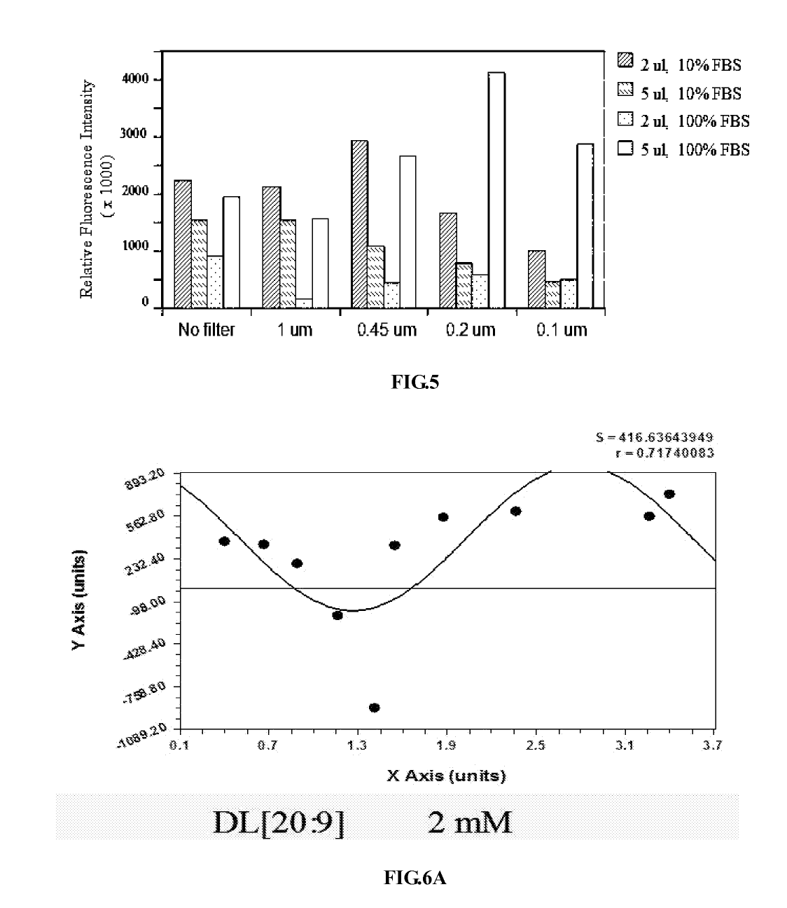

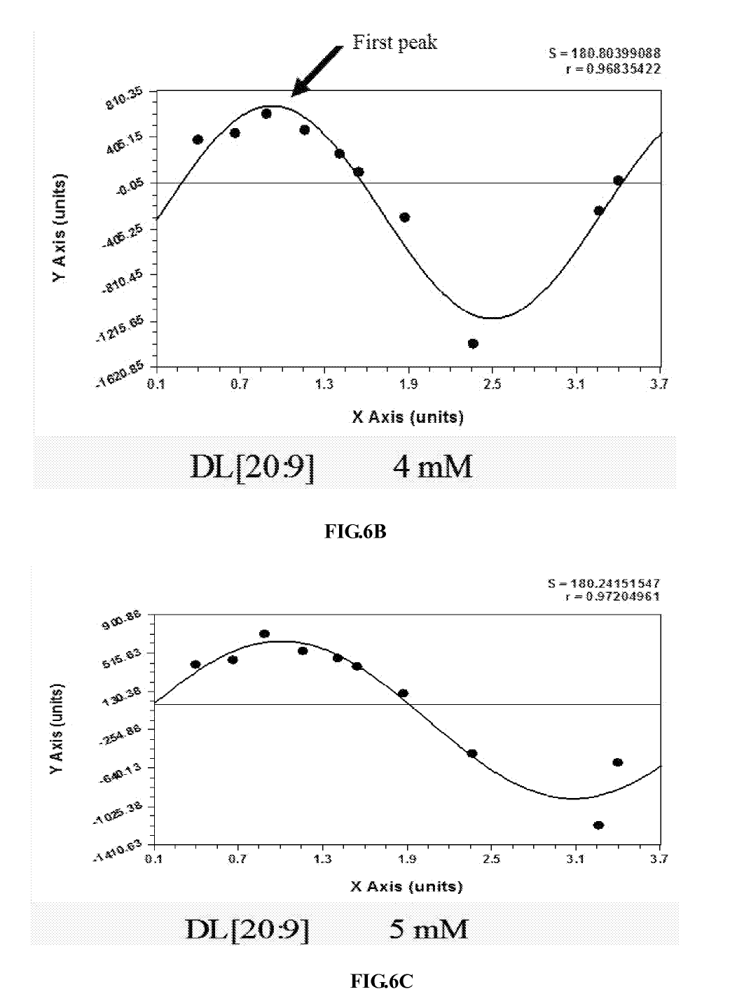

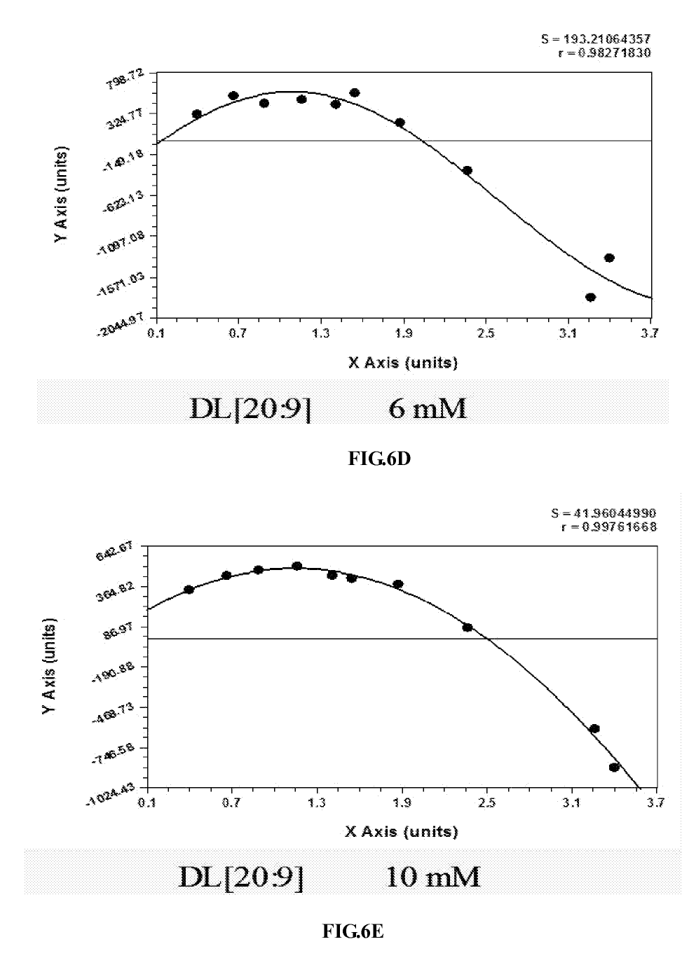

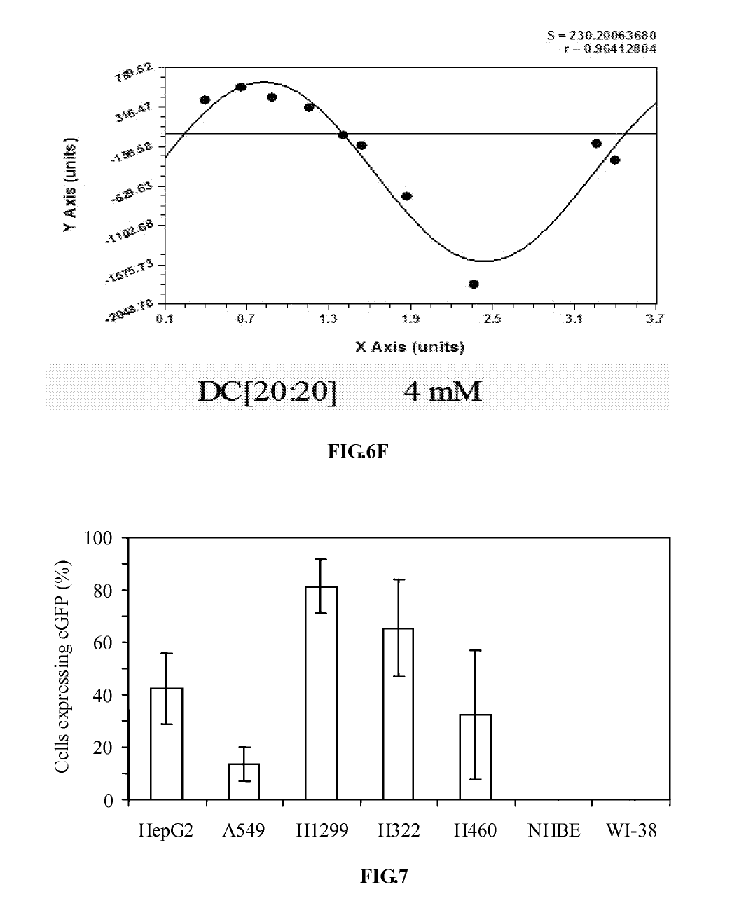

As shown in FIG. 6 OD260 value of DOTAP:Lecithin:DNA complexes vs DNA concentration matches Sinusoidal Curve Model y=a+b*cos(cx+d). Represented here is DOTAP:Lecithin. DOTAP:Lecithin liposomes with different molar ratio observe the same sinusoidal curve fit with slight shift.

### Example 5

### The Experiment of Transfection Efficiency of Complex Formed by Carrier and pFCB-eGFP Differs on Cell Lines, Said Carrier is Composed by DOTAP and Lecithin with the Molar Ratio 20:9

As shown in FIG. 7, DOTAP:Lecithin:pFCB-eGFP was used to transfect tumor cells from liver cancer and lung cancers together with normal controls NHBE and WI-38. The transfection rate for tumor cells is 15% or higher, the average transfection rate for normal control cells is less than 1%. In figure, HepG2: liver cancer. A549, H322: Adenocarcinoma. H1299, H460: large cell lung cancer. NHBE: Normal Human Bronchial Epithelial cells. WI-38: Normal Human Fibroblast cells.

### Example 6

### Preparation of DOTAP:Lecithin:DNA Complex

200 mg DOTAP and 98.8 mg Lecithin were mixed in 1 L round bottom flask with 15 ml HPLC grade Chloroform. The flask was flushed with nitrogen and attached to rotary evaporator. Evaporating chloroform for 30 min at 30° C. for 30 min under house vacuum (˜30 Hg). Remove flask from evaporator and flush with nitrogen again. Reconnect to a rotary evaporator with water bath set at 50° C. Apply house vacuum (˜30 Hg) for 15 min, detach the flask from evaporator and fill up with nitrogen. Reverse the flask with opening down and continue nitrogen flush for 15 min. dissolve the liposome film with 36 ml D5W (5% Dextrose in water) and cover flask with parafilm. Shake to make sure all material is dissolved and transfer to a 50 ml conical tube. Incubate at 50° C. for 5 min to help dissolve the liposome film completely. Pass through 0.45 um filter once and 0.1 um filters four times. Liposome thus made is 2× solutions of 8 mM of DOTAP with DOTAP:Lecithin molar ratio of 20:9. 2× liposome can be sealed under argon and stored in dark at 4° C. for 2 years without obvious oxidation.

To prepare DOTAP:Lecithin:DNA complex, DNA was dissolved in D5W at 1 μg/μl. Equal volume of DNA solution was pipette into DL liposome solution and mixed by pipette up and down for 10 times. The resulting complex should appear milky and no visible precipitant. The complex is stable at 4° C. for at least 2 months.

Plasmid DNAs were purified with Macheney-Nagel NUCLEOBOND AX columns following manufacturer's instruction. Endotoxins were further cleared twice with Endotoxin Removal Solution from Sigma-Aldrich to lower endotoxin level below 10 EU/mg measured by LAL assay from Chembrex.

DOTAP (890890P, 1,2-dioleoyl-3-trimethylammonium-propane) and egg lecithin (840051P, L-a-phosphatidylcholine) were purchased from Avanti Polar Lipids. DOTAP Methylsulfate was synthesized in house. The NUCLEOBOND AX anion exchange columns (Cat 740531.50) were purchased from Macherey-Nagel. Endotoxin Removal Solution (E4274) was purchased from Sigma-Aldrich. The endotoxin removal procedure was followed as manufacturer's suggestion.

### Example 7

### Transfection Ability DOTAP:Lecithin:pFCB-eGFP Complex Correlated with DOTAP:Lecithin Ratio and Complex Sizes

Plasmid DNA pFCB-eGFP was simplified from pUC 19 to contain pUC duplication origin, Kanamycin resistance gene, CMV promoter and CMV driven eGFP gene as reporter gene. Different molar ratio of DOTAP:Lecithin liposomes were prepared followed the protocol given in Example 6. The DOTAP:Lecithin molar ratios were adjusted to 20:7 to 20:15. DL liposomes with different molar ratio were mixed with equal volume of 1 μg/μl pFCB-eGFP. H1299 cells were plated in 12 well plates and reached 30% confluence at the time of transfection. DOTAP:Lecithin:pFCB-eGFP complex were diluted in Opti-MEM without FBS, RPMI with 10% FBS or in 100% FBS in different amounts. The mixtures were added to H1299 cells and incubated at 37 C for 4 hours. The mixtures were aspirated and replaced with fresh RPMI with 10% FBS and continue to grow for 48 hours. Pictures were taken under fluorescence microscope. The percentages of GFP expressing cells were used as measure for transfection rate. DOTAP:Lecithin complex at low molar ratio, i.e., DOTAP:Lecithin[20:7] displayed strong eGFP expression in serum free or low serum medium but eGFP expression dropped dramatically in 100% FBS. The transfection abilities of DOTAP:Lecithin complexes in 100% FBS reaches highest level at molar ratio of DOTAP vs Lecithin around 20:9 to 20:10 then the transfection efficiency decreased with addition of lecithin. DOTAP:Lecithin molar ratio at this range strikes best balance between transfection efficacy and serum protection. The same results were observed in A549 and H322 cells too.

The correlation between the transfection efficiency and particle sizes of DOTAP:Lecithin:DNA complexes were compared in a separate experiment. Particle sizes of DOTAP:Lecithin:pFCB-eGFP complex were dilated artificially by adding a irrelevant plasmid DNA—pFCB-Luc. The liposome complexes were extruded through filters with pore size of 1 μm, 0.45 μm, 0.2 μm and 0.1 μm. The size filtered liposome complexes were added to H1299 cells in RPMI with 10% FBS or 100% FBS for 4 hours. Transfection mixtures were replaced with fresh RPMI with 10% FBS and continued culture for 48 hours. Transfection efficiency was observed under fluorescence microscope. Generally, the transfection rate reached peak at 2.5 μl DOTAP:Lecithin:DNA. Smaller particles (0.2 μm or 0.1 μm) demonstrated better transfection efficiency in high serum presence. When transfected in low serum medium, 2 μl DOTAP:Lecithin[20:9]:DNA containing 1 μg DNA in 1 ml medium reached highest transfection. With complex amount increased to 5 μl per ml the transfection decreased due to the cytotoxicity. In 100% FBS, 5 μl complex in 1 ml medium gives the best transfection rate due to serum inhibition effect which also appeared to act on reducing the cytotoxicity of DOTAP:Lecithin:DNA complexes. When large particles used, the serum inhibitors neutralizes DOTAP:Lecithin:DNA complex activity. The larger the particle sizes are, the bigger the impact of serum inhibitor caused. Smaller size complexes contain more particles which saturated serum inhibitors providing more transfection active particles. Lower particle sizes below 200 nm is also help the particles better maintained in blood circulation avoiding deposition of larger particles displayed.

### Example 8

### Gene Transfer Efficacy of DOTAP:Lecithin:DNA Complex Differs with Tumor Cells and Normal Cells

Transfection efficiency of DOTAP:Lecithin:DNA complex on tumor and normal cells were compared with DOTAP:Lecithin:pFCB-eGFP. Liver cancer cell: HepG2, adenocarcinomas: A549, H322; Large cell lung cancer cells: H1299, H460 and Normal human fibroblast cell: WI-38 was from ATCC. Normal Human Bronchial Epithelial cells-NHBE was purchased from Lonza. All cells were grown overnight in 12 well plates with triplicates, medium were replaced with 2.5 μl DOTAP:Lecithin:pFCB-eGFP in 1 ml of RPMI with 10% FBS. 4 hours later, transfection medium were replaced with respective medium and cultured for 48 hours. The transfection rates were observed under fluorescence microscope. Cancer cells demonstrated greater transfection rate between 15%-80% while very few eGFP expressing cells observed in normal cells. The difference is statistically significant among normal and cancer cell groups (student's t-test, p<0.001). As we have mentioned before, there are several possible reasons behind this phenomenon. The most important ones might be the different negative natures on normal and tumor cell surfaces. All the tested tumor cells grow at least 50% faster than normal cells used, which also contributes to the high level expression of eGFP.

### Example 9

### The Comparison of Transfection Rate and Cytotoxicity

A firefly luciferase expression plasmid DNA driven by CMV promoter was mixed with different concentration of DOTAP:Lecithin. Resulting DOTAP:Lecithin:DNA complexes were used to transfect large cell lung cancer cell H1299, adenocarcinoma cell A549 and normal bronchial epithelial cell NHBE. Cells were seeded in 96 well plates in triplets. Cells were transfected 0.1 ml RPMI with 10% FBS plus 0.25 μl or 0.5 μl DOTAP:Lecithin:DNA complexes for 4 hours. 48 hours after transfection, firefly luciferase activities were measured for gene transfer efficiency. XTT assay was used for measurement of cytotoxicity of DOTAP:Lecithin:DNA complexes. XTT reading represents the percentage of alive cells in treated groups vs untreated control cells, i.e. 95 means 95% cells are alive when compared to control cells.

### Example 10

### The Expression In Vivo of DOTAP:Lecithin:pFCB-LacZ Complex

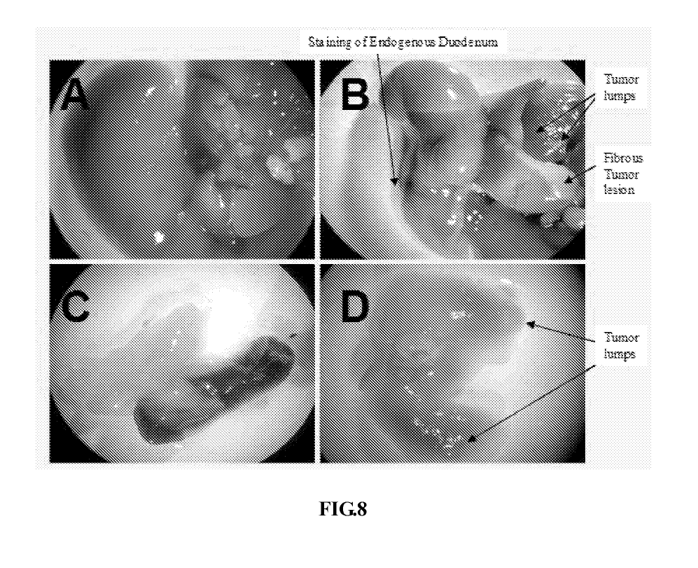

pFCB-LacZ containing CMV promoter driven LacZ gene was used for in vivo gene delivery test. DOTAP:Lecithin:pFCB-LacZ was prepared as in example 6. 1×10⁶ A549 cells were implanted in nu/nu mouse through tail vein injection. 8 weeks later, 50 μg plasmid DNA in 100 μl of DOTAP:Lecithin:pFCB-LacZ were tail vein injected in one minute interval into a control and tumor bearing mouse. Mice acted normally. 48 hours after LacZ gene administration, the mice were sacrificed and the organs were fixed in 4% paraformaldehyde with 0.1% TritonX-100 in PBS for 2-3 hrs at 4 degree. Incubate them with 1 mg/ml X-gal in Tissue stain base solution (Chemicon, Cat. # BG-8-C) in the dark humid chamber at R/T for Overnight. The tumor bearing mouse had enlarged liver containing hardened legion typical to liver cancer with pale metastatic tumor lumps spotted around lung and liver. Mouse organs from both mice were stained in Tissue stain base solution (Chemicon, BG-8-C) supplemented with 1 mg/ml x-gal for 48 hours at room temperature. The stained Endogenous duodenums were checked to blue as method control. The tumor spots in lung and liver both stained with different degrees. The tumors grown in lung is smaller than the tumors in liver, the LacZ expression level is also weaker with light blue staining. The A549 tumors grown in liver are in a much advanced stage and caused fibrous tumor lesion. The deep blue stains in liver tumors indicated the stronger LacZ expression in the liver tumor even though the tumors were all originated from the same source. The most interesting observation is the surrounding areas of fibrosis lesions were stained with light blue but the center regions of fibrosis alike liver tumors were unaffected as in FIG. 8B. Those indications are valuable for the design of therapeutic regime when concerned with different organs.

### Example 11

### DOTAP:Lecithin:p53 Complex Inhibits Subcutaneous A549 Tumor Growth in nu/nu Mice

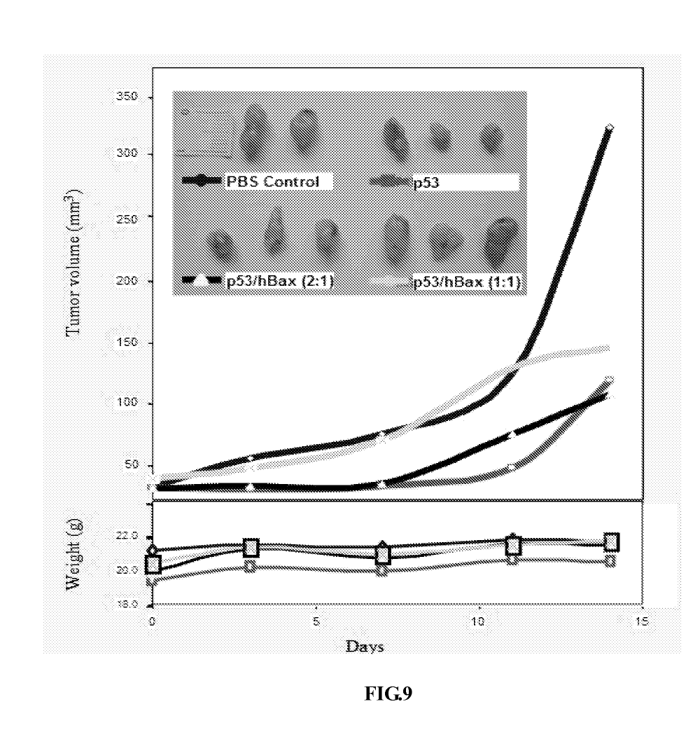

As shown in FIG. 9, to evaluate the effect of DOTAP:Lecithin:pFCB-p53 on tumor growth in vivo, A549 subcutaneous tumors were established in nu/nu mice. After tumors reached 500 mm³, tumors were separated and divided equally to 1 mm and implanted into the rear flank of nu/nu mice subcutaneously. Eleven mice were randomly divided into four treatment groups: group 1: PBS; group2: DOTAP:Lecithin:pFCB-p53 (moral ratio of DOTAP:Lecithin is 20:9); group3: DOTAP:Lecithin:[pFCB-p53(2):pFCB-hBax(1)] (moral ratio of DOTAP:Lecithin is 20:9; moral ratio of [pFCB-p53(2):pFCB-hBax(1) is 2:1); group4: DOTAP:Lecithin:[pFCB-p53(1):pFCB-hBax(1)] (moral ratio of DOTAP:Lecithin is 20:9; moral ratio of [pFCB-p53(2):pFCB-hBax(1) is 1:1). When tumors reached 30 mm³, 37 μg of plasmid DNA in total 100 μl of DNA liposome complex were slowly injected by tail vein in 1 minute. 6 doses were administered for each mouse in every two days. Tumor sizes and mouse weights were recorded every two days. 60% growth inhibition of subcutaneous tumors was achieved via systemic administration of tumor suppressor gene p53 or the combination of p53 and hBax. DOTAP:Lecithin:DNA at such dosage was well tolerated by mice. Mice in treatment groups experienced no abnormal behavior changes and average weight gain is normal compared to control group. Those preliminarily data proved the target selection of DOTAP:Lecithin:DNA and further studies for human cancer gene therapy are warranted.

1. A drug carrier for tumor-targeted therapy, consisting of two components, which is DOTAP or its analogues and lecithin or its derivatives, characterized in that: the said DOTAP or its analogues and the said lecithin or its derivatives are at molar ratio of 20:(7-13), wherein, the said DOTAP analogues is selected from DOTMA, DDAB or methyl sulfate DOTAP, the said lecithin derivatives is selected from DOPC, DOPS or DOPE.

2. A drug carrier for tumor-targeted therapy according to claim 1, characterized in that: the said DOTAP or its analogues and the said lecithin or its derivatives are at molar ratio of 20:(8-11).

3. A drug carrier for tumor-targeted therapy according to claim 2, characterized in that: the said DOTAP or its analogues and the said lecithin or its derivatives are at molar ratio of 20:9.

4. The preparation method of a drug carrier for tumor-targeted therapy according to claim 1, characterized in that: includes following steps:
a. mix above-mentioned DOTAP or its analogues and above-mentioned lecithin or its derivatives according to above-mentioned molar ratio with the organic solvent in the container, and then purge the container with nitrogen;
b. heat to 30° C. to remove chloroform by evaporation, and form liposomes film;
c. at 50° C., dissolve the liposomes film from step b with D5W solution;
d. filter one time with filter of pore size 0.45 μm, and then filter four times with filters of pore size 0.1 μm.

5. The use of a drug carrier for tumor-targeted therapy according to image transmitting apparatus according to claim 1, characterized in that: be used for cancer therapy.

6. The use of a drug carrier for tumor-targeted therapy according to image transmitting apparatus according to claim 1, characterized in that: the said carrier form complex by loading negatively charged bioactive materials.

7. The use of a drug carrier for tumor-targeted therapy according to claim 5, characterized in that: deliver negatively charged bioactive materials into live cells in vitro, ex vivo or in vivo.

8. The use of a drug carrier for tumor-targeted therapy according to claim 6, characterized in that: said negatively charged bioactive materials are DNA, RNA or oligomeric nucleic acid.

9. The use of a drug carrier for tumor-targeted therapy according to claim 6, characterized in that: said complex is mixed with protein ligands, antibodies or steroids additionally.

10. The use of a drug carrier for tumor-targeted therapy according to claim 6, characterized in that: the range of particle size of the said complex is 90 nm-250 nm.

11. The use of a drug carrier for tumor-targeted therapy according to claim 6, characterized in that: the said complex is used for combination with chemotherapy, radiation therapy or both.

12. The use of a drug carrier for tumor-targeted therapy according to claim 6, characterized in that: prepare the said complex in form of injection or aerosol.

13. The use of a drug carrier for tumor-targeted therapy according to claim 6, characterized in that: carrier loading negatively charged bioactive materials to achieve the maximal packaging capacity of the carrier, the method is that, the correction between absorbance of said complex at the wavelength of 260 nm and the concentration of said negatively charged bioactive materials is in line with Sinusoidal Curve Fit model between the carrier and the concentration of said negatively charged bioactive materials; The first peak of sinusoidal curve indicates the maximal packaging capacity of the carrier.

14. The use of a drug carrier for tumor-targeted therapy according to claim 6, characterized in that: the lowest cytotoxicity of the complex is achieved, the method is that: net increased value of complex optical absorbance at 260 nm is correlated with cytotoxicity of the complexes, the DNA concentration at the first peak of OD260 sinusoidal curve of the complexes indicates the concentration for lowest cytotoxicity of complexes in vitro and in vivo.

15. The use of a drug carrier for tumor-targeted therapy according to claim 6, characterized in that: to achieve the highest transfection capability, the method is that: net increased value of complex optical absorbance at 260 nm is positively correlated with transfection capability of the complexes, the DNA concentration at the first peak of OD260 sinusoidal curve of the complexes indicates the concentration of highest transgene expression of complexes in vitro and in vivo.

16. The use of a drug carrier according to claim 1 for manufacturing a complex for the treatment of cancer.
